(19) United States
(12) Patent Application Publication
(10) Pub. No.: US 2008/0033259 A1
(43) Pub. Date: Feb. 7, 2008

(54) PORTABLE MEDICAL DEVICE FOR AUTOMATIC ELECTRICAL COHERENCE ANALYSIS INSIDE A PATIENT

(75) Inventors: Mario Manto, Braine-le-Chateau (BE); Jose Pons Rovira, Madrid (ES); Ramon Ceres Ruiz, Madrid (ES); Fernando Brunetti, Madrid (ES)

Correspondence Address: KNOBBE MARTENS OLSON & BEAR LLP, 2040 MAIN STREET, FOURTEENTH FLOOR, IRVINE, CA, 92614, US

(73) Assignees: UNIVERSITE LIBRE DE BRUXELLES, Brussels (BE); INSTITUTO DE AUTOMATICA INDUSTRIAL, CSIC, Madrid (ES)

(21) Appl. No.: 11/608,772

(22) Filed: Dec. 8, 2006

(30) Foreign Application Priority Data
Jun. 9, 2005 (BE) PCT/BE05/00095
Jun. 9, 2004 (EP) 04447141.5

(51) Int. Cl. A61B 5/00

(52) U.S. Cl. 600/301

(57) ABSTRACT

A portable medical device for automatically monitoring of electrical activity of target areas inside a patient is described. The medical device is based on a wireless communication network and includes a measuring unit that measures electrical signals at the surface of two target areas and a processing unit connected to the measuring unit. The processing unit includes a slave unit and a master unit for collecting and analyzing the measured electrical signals and presenting the result of the analysis for a clinician. The processing unit also includes a visualization unit connected to the master unit and an optional basic unit for at least presenting the result of the electrical signals and analysis, where the master unit and the basic unit perform an analysis of the electrical signals including an analysis of the coherence between the first and second sets of electrical signals as a function of the monitoring frequency.

### RELATED APPLICATIONS

This is a continuation application under 35 U.S.C. §120 of WO 2005/120347 A1, filed as PCT/BE2005/000095 on Jun. 9, 2005, which is hereby incorporated by reference.

### BACKGROUND

1. Field of the Invention

The present invention concerns the field of medical devices.

More precisely, the present invention is related to a new device for analysing physiological signals, and preferably tremors, inside a patient. State of the Art

Human tremor corresponds to a rhythmic activity of muscles and is the most common movement disorder. Recent studies have shown that 3 to 4% of the population aged higher than 60 years present a tremor in limbs and/or head.

Different medical devices have already been proposed in prior art for monitoring physiological signals inside a patient, and namely for monitoring electromyography signals. Examples of commercially available telemetry devices are MyoMonitor (EMG Systems), MT8 Telemetry system (MIE Medical Research Ltd) and MESPEC 4000 Telemetry (MEGA Electronics).

Tools for monitoring signals during daily life have also been proposed. For example, Keijsers N. L. et al. (“Online monitoring of dyskinesia in patients with parkinson's disease”, IEEE EMB Magazine, vol. May/June, pp. 96-103, 2003) have proposed accelerometers for online monitoring of dyskinesia.

Most of the devices use sensors for measuring tremor signals in the form of surface electrodes or needle electrodes to be placed at specific target areas on the patient, including brain and muscles.

However, these medical devices present several drawbacks which limit their use. Indeed, these devices do not automatically provide relevant informations about the signals measured which could be instantaneously used as such by a doctor for a diagnosis. On the contrary, the data collected by these devices require further investigations under the control of very specialized people trained therefore in dedicated centers. In addition, the size of these devices is such that signal monitoring at the bed of the patient (bedside examination) is impossible and the displacement of the patient is necessary.

In other words, the prior art devices provide a signal monitoring which is time-consuming, expensive, and uncomfortable for the patient.

There is thus a rather important need for an enhanced medical device, and namely a medical device adapted to tremor analysis.

### SUMMARY

A medical device is provided for monitoring physiological signals in a patient, and especially tremor signals, which does not present the drawbacks of the prior art devices as disclosed hereabove.

In particular, a portable medical device is provided which is easy to use, of moderate cost and which provides automatically, and instantaneously if required, a relevant analysis of the measured signals which could be used directly by a doctor for a diagnostic. Furthermore, a medical device is provided which is non invasive.

In one embodiment there is a portable medical device for automatic monitoring of the electrical activity of target areas inside a patient, the medical device comprising a measuring unit comprising at least a first sensor and a second sensor configured to measure, as a function of a monitoring frequency, a first set of electrical signals at the surface of a first patient target area, and a second set of electrical signals at the surface of a second patient target area, respectively; a processing unit connected to the measuring unit, the processing unit comprising a slave unit configured to collect the first set of electrical signals measured by the measuring unit; a master unit connected to the slave unit and configured to receive the first set of electrical signals collected by the slave unit, to collect the second set of electrical signals measured by the second sensor (and to analyze the first set and the second set of electrical signals thus collected); a visualization unit, such as a LCD display, connected to the master unit and configured to at least present the result of the electrical signals analysis performed by the master unit, and possibly a basic unit connected to the master unit and configured to receive and record the first set and the second set of electrical signals collected by the master unit (and to analyze the first set and second set of electrical signals and to present the result of the obtained analysis under appropriate form for a clinician), wherein the master unit and the possible basic unit are configured to analyze the electrical signals, comprising an analysis of coherence between the first and second sets of electrical signals as a function of the monitoring frequency, and wherein the device further comprises a wireless communication unit to provide a wireless communication connection between the different units of the device.

Preferably, the electrical signals correspond to tremor signals.

Advantageously, the sensors are selected from the group consisting of electromyography sensors, electro-encephalography sensors and cortical sensors.

Preferably, the sensors are biomechanical sensor electrodes, such as surface electrodes, (intra-muscular) needle electrodes or micro-electrodes.

Advantageously, the present medical device performs inter-muscular or intramuscular tremor coherence analysis.

Preferably, the medical device is adapted for working at monitoring frequencies comprised between about 0.1 Hz and about 30 Hz, preferably between about 3 Hz and about 16 Hz, and more preferably between about 3 Hz and about 12 Hz.

Preferably, the basic unit comprises a configuration tool to set up the configuration parameters of the device in operating conditions, the configuration parameters comprising the sampling frequency at which the master and slave units operate and the monitoring frequencies.

Advantageously, the wireless communication unit of the present medical device uses Bluetooth technology.

In another embodiment, there is a portable medical device for automatic monitoring of electrical activity of target areas inside a patient, the medical device comprising a measuring unit comprising at least a first sensor and a second sensor configured to measure a first set of electrical signals at the surface of a first patient target area, and a second set of electrical signals at the surface of a second patient target area, respectively; and a processing unit connected to the measuring unit, the processing unit comprising subunits configured to collect the first set of electrical signals measured by the first sensor and the second set of electrical signals measured by the second sensor, to record the first set and the second set of electrical signals, to analyze the first and second sets of electrical signals and to present the result of the analysis under appropriate form for a clinician, wherein the analysis comprises analyzing coherence between the first and second sets of electrical signals as a function of monitoring frequency, and wherein the medical device further comprises a wireless communication unit to provide a wireless communication connection between the different units of the medical device.

The processing unit additionally may comprise a visualisation subunit configured to at least present the result of the electrical signals analysis. The electrical signals may correspond to tremor signals. The sensors may be selected from the group consisting of electromyography sensors, electro-encephalography sensors and cortical sensors. The sensors may be biomechanical sensor electrodes, where the electrodes may be selected from the group consisting of surface electrodes, needle electrodes and micro-electrodes. The medical device may perform inter-muscular tremor coherence analysis. The medical device may operate at monitoring frequencies comprised between about 0.1 Hz and about 30 Hz. The processing unit may comprise a configuration tool to set up the configuration parameters of the medical device in operating conditions, the configuration parameters comprising the sampling frequency at which at least a portion of the subunits operate and the monitoring frequencies.

The use of the present medical device also concerns use as a tool for characterizing tremors related to specific diseases.

Advantageously, the present medical device may also be used as a tool for testing the effect of drugs on tremors.

However, it has to be noted, in certain embodiments, that the medical device provides physical data corresponding to intermediate results concerning the tremor which do not on their own enable a decision to be made by the doctor or surgeon on a diagnostic or treatment.

### BRIEF DESCRIPTION OF THE DRAWINGS

FIG. 1 represents a schematic view of the different units of a device according to one embodiment.

FIG. 2a shows an example of application of the device for monitoring intralimb tremor coherence.

FIG. 2b-2c and FIG. 3 show an example of application of the device for monitoring interlimb tremor coherence.

FIG. 4 gives an example of EMG signals as acquired with one embodiment of a medical device.

### DETAILED DESCRIPTION OF CERTAIN EMBODIMENTS

The following detailed description of certain embodiments presents various descriptions of specific embodiments of the invention. However, the invention can be embodied in a multitude of different ways as defined and covered by the claims. In this description, reference is made to the drawings wherein like parts are designated with like numerals throughout.

The terminology used in the description presented herein is not intended to be interpreted in any limited or restrictive manner, simply because it is being utilized in conjunction with a detailed description of certain specific embodiments of the invention. Furthermore, embodiments of the invention may include several novel features, no single one of which is solely responsible for its desirable attributes or which is essential to practicing the inventions herein described.

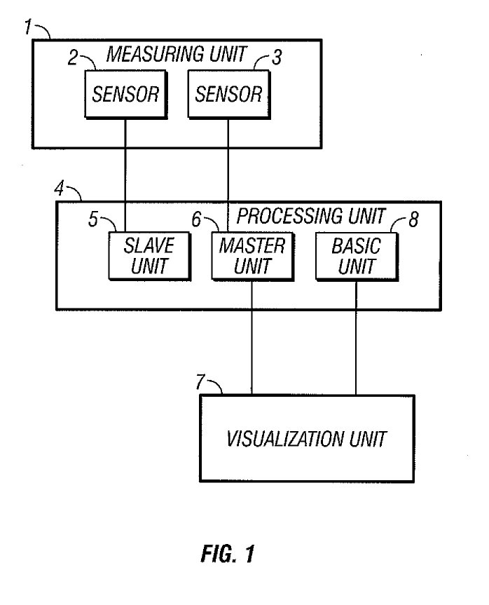

FIG. 1 represents a diagram, wherein the different elements of a portable device according to certain embodiments and their relationship with one another are represented.

As shown in FIG. 1, the medical device comprises a measuring unit 1 comprising at least a first sensor 2 and a second sensor 3. The sensors 2,3 may be electromyography sensors, electro-encephalography sensors, or cortical sensors, and preferably take the form of electrodes possibly connected to a battery (15) and combined with low-pass or high-pass filters. The electrodes may be for example surface electrodes or (intramuscular) needle electrodes possibly bound to the skin 12 by an interface adhesive 13 or inserted in the muscles 14 (at specific locations 10,11).

Furthermore, the device may comprise electromyographic amplifiers that advantageously improve the signal/noise ratio.

Both first and second sensors 2,3 are able in operating conditions to measure electrical activities at monitoring frequencies comprised between about 0.1 Hz and about 30 Hz, preferably between about 3 Hz and about 16 Hz, and more preferably between about 3 Hz and about 12 Hz.

The medical device also comprises a processing unit 4 connected to the measuring unit 1 by means of a wireless communication network using, for example, Bluetooth technology or HomeRF or WiFi technology or IrDA technology.

The function of the processing unit 4 is to process (amplification, filtration, analysis) in operating conditions the data concerning the electrical signals measured and transmitted by the sensors 2,3 in such a way as to provide relevant information directly available to a clinician (obtained coherence value is computed, stored and displayed) but it can also be monitored and further analyzed off-line by means of additional displaying hardware and software.

More precisely, the processing unit 4 comprises a slave unit 5 which is connected to the first sensor 2 for collecting the first set of electrical signals measured by the first sensor 2.

The slave unit 5 is connected to a master unit 6 in such a way that the master unit 6 may receive in operating conditions the first set of electrical signals collected by the slave unit 4.

The master unit 6 is also connected to the second sensor 3 for collecting the second set of electrical signals measured by the second sensor 3.

In addition, the master unit 6 is configured so as to allow an analysis of the first set and the second set of electrical signals it has thus collected.

A basic unit 8 is optionally connected to the master unit 6 for receiving and recording the first set and the second set of electrical signals collected by the master unit 6, analyzing them and presenting the result of the analysis under appropriate form for the clinician.

In other words, both the master unit 6 and the basic unit 8 are able to perform the analysis of the first set and the second set of electrical signals collected by the master unit 6.

The medical device further comprises a visualisation unit 7, such as a LCD display (e.g., 8 columns×2 lines character display), connected to the master unit 6 and possibly to the basic unit 8 which is able on demand to present the result of the electrical signals analysis performed by the master unit 6.

In the medical device, the master unit 6 and possibly the basic unit 8 are configured so as to allow an analysis of the electrical signals comprising the analysis of a coherence between the first and second sets of electrical signals as a function of a monitoring frequency.

The processing of the two sets of electrical signals by the master unit 6 and the basic unit 8 comprises the calculation of the function of coherence obtained by a configuration of these units (6 and 8) that apply the following coherence algorithm. Coherence is a bounded measure of linear correlation and is defined as follows for two signals x and y:

$${γ}_{x y} ( f ) = \frac{{G}_{x y}}{\sqrt{{G}_{x x} ( f ) {G}_{y y} ( f )}} \qquad ( 1 )$$

wherein f denotes the considered frequency and Gxy the complex cross power spectrum stated by:

$${G}_{x y} ( f ) = {\int }_{- ∞}^{+ ∞} {R}_{x y} ( τ ) {ⅇ}^{j 2 π f τ} ⅆ τ , \qquad ( 2 )$$

and is the Fourier transform of the cross correlation function:
R_{xy}(τ)=E[x(t)y(t+τ)]. (3)
wherein x and y (EMG signals) are real and E[ ] denotes the mathematical expectation.

Coherence is equal to one (1) when one signal is a linear function of the second signal, and coherence is equal to zero (0) in the case of linear independence.

It should be noted that the dimensions, compositions and weight of the different units (elements) of the present medical device are such that it is portable and it can easily be placed in the pocket of clothes.

The device may also comprise one or more (electromyographic) amplifiers for amplifying adequately the acquired signals and improving signal/noise ratio.

The communication network between the different elements of the device is of a wireless type, and uses, for example, the Bluetooth technology or the HomeRF or the WiFi technology or the IrDA technology.

As a consequence, the medical device has the advantage of being independent from any electrical installation and could be used in extreme conditions, as for example for quick examination of road accident victims.

As another consequence, the medical device could be easily used during bedside examination of patients, thereby being more comfortable for the patient than the prior art devices.

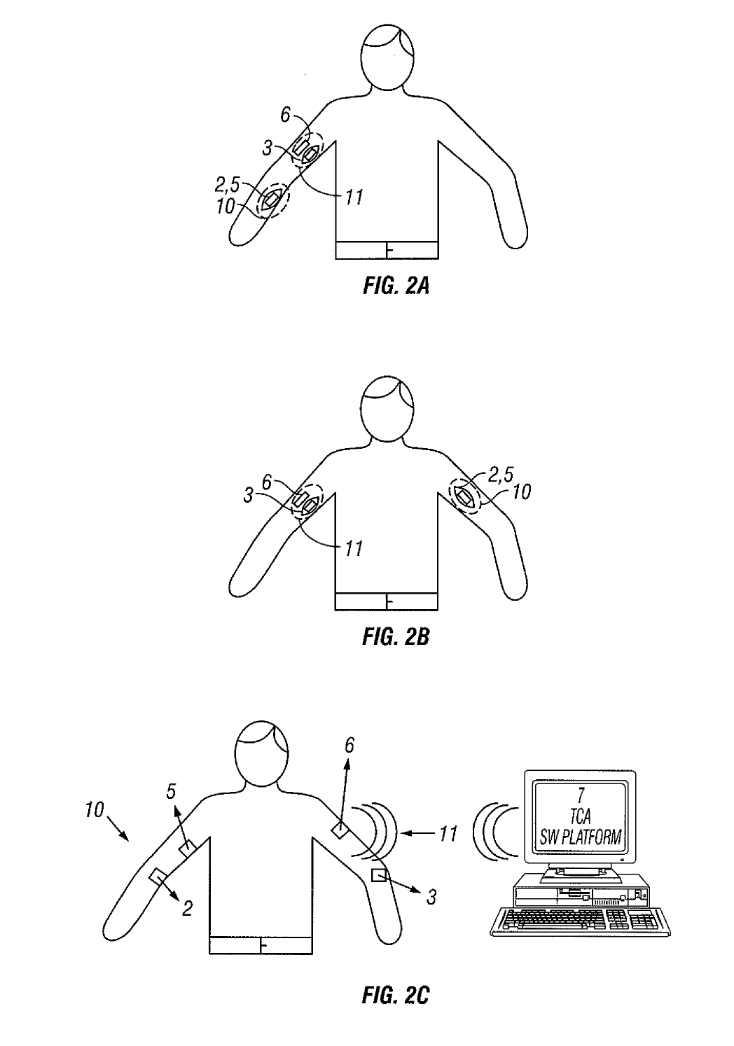

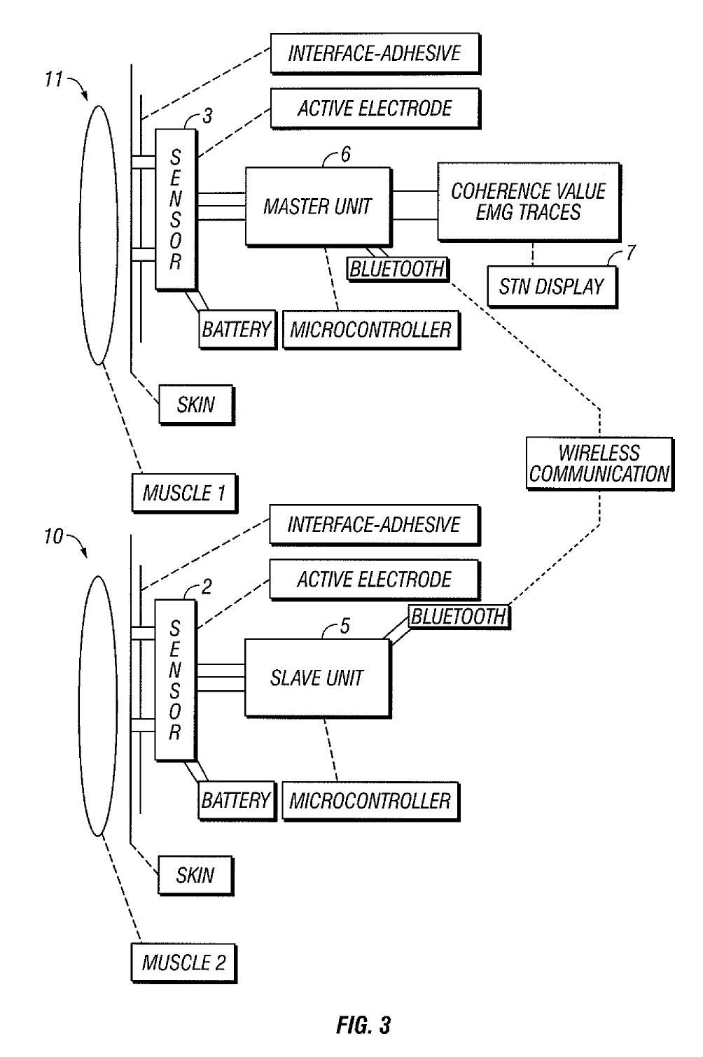

FIG. 2a, FIGS. 2b-2c and FIG. 3 illustrate two examples of possible application of such a medical device. As shown in FIG. 2a, the sensors can be placed on two different areas 10,11 located on the same upper limb of a patient for monitoring intralimb muscular tremor coherence. The sensors 2,3 may also be placed on two different areas 10,11 located each on a different upper limb of a patient for monitoring interlimb muscular tremor coherence, as illustrated in FIGS. 2b-2c and FIG. 3.

However, it is important to note that the present medical device is not limited to the illustrations. Other applications of the medical device could be envisaged by the person skilled in the art.

For example, the sensors may be adapted for a fixation on other patient target areas such as other muscles or the brain.

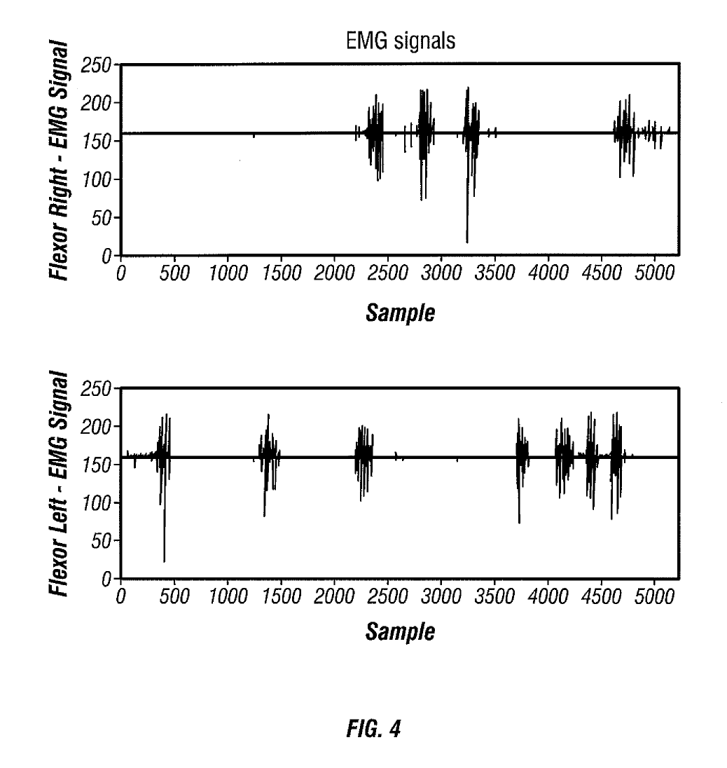

FIG. 4 gives an example of EMG signals as acquired with one embodiment of the medical device. The technical characteristics of the medical device in one embodiment are the following:

master unit:
3.6V Lithium Ion rechargeable battery
Microcontroller 3.3V
3.3V voltage regulator
Signal Conditioning block.
Communication with the Bluetooth module using serial port at 115 kbps
7.32 MHz system clock.
Sampling Frequency: 512 Hz.

slave unit: idem as for the master unit

signal conditioning module (in both main and slave units):
Signal Amplification.
Low pass filter (400 Hz).
Signal Rectification.
Diode clipping circuit.

Computer (basic unit 8):
Windows operating system.
Developed TCA software.
RS232-Serial port.
Bluetooth module.

Beside muscular tremor coherence analysis, the medical device may also be configured in such a way as to allow monitoring of coherence between other physiological signals detectable through electrical activity measurement, such as cortico-cortical coherence analysis and cortico-muscular coherence analysis.

Furthermore, the medical device could be used not only as a routine tool usable by a doctor during its daily practice, but also as a research tool for different investigations.

For example, the medical device could be used as a tool to investigate in a patient if there is a unique generator that generates tremors or if there are several tremor generators interacting with each other. From that point of view, the medical device could be considered as a useful tool providing data which may help doctors or clinicians to establish a better diagnostic (analysis of the electromyographic signals in real time with a direct display of the coherence value).

The present medical device could also be used by clinicians or pharmaceutical companies to test the effects of drugs or other kinds of stimuli on the electrical activity (changes, events, actions) by a direct assessment of the effect of the drugs and stimuli on the coherence between electromyographic signals.

The present medical device also has useful applications such as:
assessment of deep brain stimulation (stimulation of thalamic nuclei Vim, stimulation of subthalamic nuclei STN) on the coherence;
on line direct estimation of the value of intermuscular coherence in orthostatic tremor;
detection of the shift from a low coherence to a high coherence, as observed in high-frequency synchronous discharges;
epidemiological analysis of intermuscular coherence in Parkinson's disease and essential tremor;
investigations of the effects of extra-load on the coherence in rest, postural and kinetic tremor;
investigations of the effects of fatigue or vibrations on the coherence between muscles.
estimation of coherence between two or more muscle fibers from one or different muscles, whose discharges are preferably recorded using micro-electrodes.
   Conclusion

While specific blocks, sections, devices, functions and modules may have been set forth above, a skilled technologist will realize that there are many ways to partition the system, and that there are many parts, components, modules or functions that may be substituted for those listed above.

While the above detailed description has shown, described, and pointed out the fundamental novel features of the invention as applied to various embodiments, it will be understood that various omissions and substitutions and changes in the form and details of the system illustrated may be made by those skilled in the art, without departing from the intent of the invention.

What is claimed is:

1. A portable medical device for automatic monitoring of electrical activity of target areas inside a patient, the medical device comprising:
a measuring unit comprising at least a first sensor and a second sensor configured to measure, as a function of a monitoring frequency, a first set of electrical signals at the surface of a first patient target area, and a second set of electrical signals at the surface of a second patient target area, respectively; and
a processing unit connected to the measuring unit, the processing unit comprising:
(i) a slave unit configured to collect the first set of electrical signals measured by the measuring unit;
(ii) a master unit connected to the slave unit and configured to receive the first set of electrical signals collected by the slave unit, to collect the second set of electrical signals measured by the second sensor, and to perform analysis of the first and second sets of electrical signals;
(iii) a visualisation unit connected to the master unit and configured to at least present the result of the electrical signals analysis performed by the master unit; and
(iv) a basic unit connected to the master unit and configured to receive and record the first set and the second set of electrical signals collected by the master unit, to analyze the first and second sets of electrical signals and to present the result of the analysis under appropriate form for a clinician,
wherein the master unit and the basic unit are configured to analyze the electrical signals, comprising an analysis of coherence between the first and second sets of electrical signals as a function of the monitoring frequency, and wherein the device further comprises a wireless communication unit to provide a wireless communication connection between the different units of the device.

2. The medical device according to claim 1, wherein the electrical signals correspond to tremor signals.

3. The medical device according to claim 1, wherein the sensors are selected from the group consisting of electromyography sensors, electro-encephalography sensors and cortical sensors.

4. The medical device according to claim 1, wherein the sensors are biomechanical sensor electrodes.

5. The medical device according to claim 1, wherein the medical device performs inter-muscular tremor coherence analysis.

6. The medical device according to claim 1, wherein the medical device operates at monitoring frequencies between about 0.1 Hz and about 30 Hz.

7. The medical device according to claim 1, wherein the basic unit comprises a configuration tool to set up the configuration parameters of the device in operating conditions, the configuration parameters comprising the sampling frequency at which the master and slave units operate and the monitoring frequencies.

8. The medical device according to claim 1, wherein the wireless communication unit uses Bluetooth technology.

9. The medical device according to claim 1, wherein the visualisation unit is a LCD display.

10. The medical device according to claim 4, wherein the electrodes are selected from the group consisting of surface electrodes, needle electrodes and micro-electrodes.

11. The medical device according to claim 6, wherein the medical device operates at monitoring frequencies between about 3 Hz and about 12 Hz.

12. A portable medical device for automatic monitoring of electrical activity of target areas inside a patient, the medical device comprising:
a measuring unit comprising at least a first sensor and a second sensor configured to measure a first set of electrical signals at the surface of a first patient target area, and a second set of electrical signals at the surface of a second patient target area, respectively; and
a processing unit connected to the measuring unit, the processing unit comprising subunits configured to collect the first set of electrical signals measured by the first sensor and the second set of electrical signals measured by the second sensor, to record the first set and the second set of electrical signals, to analyze the first and second sets of electrical signals and to present the result of the analysis under appropriate form for a clinician,
wherein the analysis comprises analyzing coherence between the first and second sets of electrical signals as a function of monitoring frequency, and wherein the medical device further comprises a wireless communication unit to provide a wireless communication connection between the different units of the medical device.

13. The medical device according to claim 12, wherein the processing unit additionally comprises a visualisation subunit configured to at least present the result of the electrical signals analysis.

14. The medical device according to claim 12, wherein the electrical signals correspond to tremor signals.

15. The medical device according to claim 12, wherein the sensors are selected from the group consisting of electromyography sensors, electro-encephalography sensors and cortical sensors.

16. The medical device according to claim 12, wherein the sensors are biomechanical sensor electrodes.

17. The medical device according to claim 12, wherein the medical device performs inter-muscular tremor coherence analysis.

18. The medical device according to claim 12, wherein the medical device operates at monitoring frequencies between about 0.1 Hz and about 30 Hz.

19. The medical device according to claim 12, wherein the processing unit comprises a configuration tool to set up the configuration parameters of the medical device in operating conditions, the configuration parameters comprising the sampling frequency at which at least a portion of the subunits operate and the monitoring frequencies.

20. The medical device according to claim 16, wherein the electrodes are selected from the group consisting of surface electrodes, needle electrodes and micro-electrodes.
